(19) United States
(12) Patent Application Publication
(10) Pub. No.: US 2009/0117552 A1
(43) Pub. Date: May 7, 2009

(54) Method for Detection and Quantification of Target Nucleic Acids in a Sample

(75) Inventors: Ying Wu, Nijmegen (NL); Marinus Gerardus Johannes Van Beuningen, Oss (NL)

Correspondence Address: AMSTER, ROTHSTEIN & EBENSTEIN LLP, 90 PARK AVENUE, NEW YORK, NY, 10016, US

(73) Assignee: PAMGENE BV, Hertogenbosch (NL)

(21) Appl. No.: 11/922,522

(22) PCT Filed: Jul. 5, 2006
(86) PCT No.: PCT/EP2006/006542

§ 371 Date: Oct. 14, 2008

(30) Foreign Application Priority Data
Jul. 7, 2005 (EP) 05447165.1

(51) Int. Cl. C12Q 1/68; C40B 40/08

(52) U.S. Cl. 435/6; 506/17

(57) ABSTRACT

The present invention relates to methods for multiplex detection and quantification of target nucleic acid sequences in a sample comprising the steps of: (i) providing a solid support having immobilized thereon an array of detector oligonucleotides, wherein said array of detector oligonucleotides is designed by random selection of non-eukaryotic genomic sequences followed by random selection of oligonucleotide sequences and subsequent conversion of these oligonucleotide sequences such that these are composed of only three types of nucleotides; (ii) providing a sample having added thereto a fixed amount of control nucleic acid of known sequence; (iii) contacting said sample with at least two probes that hybridise to adjacent sites of a target sequence under conditions favouring hybridisation between the sample nucleic acids and the said at least two probes, wherein, a) a first probe is composed of a 5′ end sequence part for hybridisation to a PCR primer and a 3′ end sequence part for hybridisation to the target nucleic acid; and b) a second probe is composed of a 5′ end sequence part for hybridisation to the target nucleic acid, and a 3′ end sequence part for hybridisation with a PCR primer, and c) an intermediate sequence is present in between said 5′ and 3′ end sequence parts of said first or second probe; and d) said second probe is characterized by having 5′ phosphate group allowing ligation with a 3′ hydroxyl group at the said first probe forming a ligation-mediated probe; (iv) ligation of the said hybridised first and second probes to form ligation-mediated probes; (v) contacting a set of detectable labelled PCR primers with the ligation-mediated probes allowing amplification thereof; (vi) detection and quantification of sample nucleic acids via hybridisation of the said intermediate parts within the amplified ligation-mediated probes onto the array of detector oligonucleotides provided in The present invention also relates to the use of said methods as well as microarrays and kits for performing said methods.

### FIELD OF THE INVENTION

The present invention relates to a method for detection and quantification of nucleic acids and nucleic acid variations in a sample. A particular aspect of the invention relates to an assay for detection of SNPs (single nucleotide polymorphisms) and nucleic acid copy numbers.

### BACKGROUND

Spontaneous, induced and hereditary changes or mutations to the genetic material (usually DNA or RNA) of cells in multicellular organisms such as humans can cause disease. Mutations can affect human health, causing disease by disrupting a cell's normal biological functions. Changes in the DNA caused by mutation can cause errors in protein sequence, creating partially or non-functional proteins.

To function correctly, each cell depends on thousands of proteins to function in the right places at the right times. Sometimes, gene mutations prevent one or more of these proteins from functioning correctly, causing malfunction or loss of a necessary protein. When a mutation alters a protein that plays a critical role in the body, a medical condition or genetic disorder can result.

The most common type of variation in the human genome is the single nucleotide polymorphism (SNP or ‘snip’), where a single base differs between individuals.

The effect of a single SNP on a gene may not be large—perhaps influencing the activity of the encoded protein in a subtle way—but even subtle effects can influence susceptibility to common diseases, such as heart disease or Alzheimer's disease.

It is clear that SNPs and other sequence variations such as point mutations, deletions, insertions, inversions, rearrangements, alternative exons and the like, are of great value to biomedical research and in developing pharmacy products.

Variations in a nucleic acid sample can be detected by the multiplex ligation-dependent probe amplification (MLPA) technique. This technique as published by Schouten et al. (Nucleic Acid Research, 2002, Vol. 30, No. 12e57) is based on the PCR amplification of ligation-mediated probes wherein each probe consists of two oligonucleotides that hybridise to adjacent sites of a target sequence and are subsequently ligated. One of the oligonucleotides comprises a stuffer sequence of different length within different oligonucleotides allowing identification of the nucleic acid variation via acrylamide gel separation of the amplification products.

A variation to the above technique was introduced as disclosed in WO 2004/053105 by an oligonucleotide array based technique wherein part of a first portion of a target nucleic acid hybridises to an immobilised capture oligonucleotide and part of a second portion of the target nucleic acid hybridises to a detector probe.

Another alternative technique is disclosed in WO 2005/054505 and relates to the conversion of the ligated oligonucleotides into easy to purify species in combination with an oligonucleotide array platform.

Further to many other methods developed in the art for the detection and/or quantification of nucleic acid variations in a sample, it will be well-appreciated that there is a continuing need for improved or alternative assays with high efficiency and through-put.

The present invention provides a method for multiplex detection and quantification of target nucleic acid sequences in a sample which addresses this need. In particular, it is the aim of the present invention to provide a method for multiplex detection and quantification of target nucleic acid sequences in a sample with high specificity and minimised cross-homology.

### SUMMARY OF THE INVENTION

The present invention relates to a method for multiplex detection and quantification of target nucleic acid sequences in a sample (see FIGS. 2 and 3) comprising the steps of:
(i) providing a solid support having immobilized thereon an array of detector oligonucleotides, wherein each detector oligonucleotide is composed of only three types of nucleotides;
(ii) providing a sample having added thereto a fixed amount of control nucleic acid of known sequence;
(iii) contacting said sample with at least two probes that hybridise to adjacent sites of a target sequence under conditions favouring hybridisation between the sample nucleic acids and the said at least two probes, wherein,
   (a) a first probe is composed of a 5′ end sequence part for hybridisation to a PCR primer and a 3′ end sequence part for hybridisation to the target nucleic acid; and
   (b) a second probe is composed of a 5′ end sequence part for hybridisation to the target nucleic acid, and a 3′ end sequence part for hybridisation with a PCR primer; and
   (c) an intermediate sequence is present in between said 5′ and 3′ end sequence parts of said first or second probes; and
   (d) said second probe is characterized by having a 5′ phosphate group allowing ligation with a 3′ hydroxyl group at the said first probe forming a ligation-mediated probe;
(iv) ligation of the said hybridised first and second probes to form ligation-mediated probes;
(v) contacting a set of detectable labelled PCR primers with the ligation-mediated probes allowing amplification thereof;
(vi) detection and quantification of sample nucleic acids via hybridisation of the said intermediate parts within the amplified ligation-mediated probes onto the array of detector probes provided in (i).

In particular, the present invention relates to a method for multiplex detection and quantification of target nucleic acid sequences in a sample (see FIGS. 2 and 3) comprising the steps of:
(i) providing a solid support having immobilized thereon an array of detector oligonucleotides, wherein said array of detector oligonucleotides is designed by random selection of non-eukaryotic genomic sequences followed by random selection of oligonucleotide sequences and subsequent conversion of these oligonucleotide sequences such that these are artificial sequences composed of only three types of nucleotides;
(ii) providing a sample having added thereto a fixed amount of control nucleic acid of known sequence;
(iii) contacting said sample with at least two probes that hybridise to adjacent sites of a target sequence under conditions favouring hybridisation between the sample nucleic acids and the said at least two probes, wherein,
   (a) a first probe is composed of a 5′ end sequence part for hybridisation to a PCR primer and a 3′ end sequence part for hybridisation to the target nucleic acid; and
   (b) a second probe is composed of a 5′ end sequence part for hybridisation to the target nucleic acid, and a 3′ end sequence part for hybridisation with a PCR primer; and
   (c) an intermediate sequence is present in between said 5′ and 3′ end sequence parts of said first or second probes; and
   (d) said second probe is characterized by having a 5′ phosphate group allowing ligation with a 3′ hydroxyl group at the said first probe forming a ligation-mediated probe;
(iv) ligation of the said hybridised first and second probes to form ligation-mediated probes;
(v) contacting a set of detectable labelled PCR primers with the ligation-mediated probes allowing amplification thereof;
(vi) detection and quantification of sample nucleic acids via hybridisation of the said intermediate parts within the amplified ligation-mediated probes onto the array of detector probes provided in (i).

The method of the present invention comprises the use of a solid support having immobilized thereon an array of detector oligonucleotides, wherein each detector oligonucleotide is composed of only three types of naturally occurring nucleotides.

Within the present invention, the totality of detector probes for a single array is designed in a unique way, completing a series of steps departing from non-eukaryotic genome sequences to arrive at a set of unique artificial sequences meeting a range of criteria in order to arrive at arrays of detector probes with minimised secondary structure formation and no cross-homology with target sequences in a sample.

The method of the present invention further provides for the quantification of target nucleic acid sequences in a sample by the introduction to the said sample of a fixed amount of control nucleic acid of known sequence; allowing the application of a normalization algorithm.

The method of the present invention allows the multiplicity of probes used in an experiment to be replaced by another multiplicity of probes without the need of developing a different array of detector oligonucleotides.

### DETAILED DESCRIPTION OF THE INVENTION

The present invention relates to the detection and quantification of target nucleic acids in a sample.

In the present specification and the appended claims, the singular forms “a”, “an”, and “the” include plural references unless the context clearly dictates otherwise. Unless defined otherwise, all technical and scientific terms used herein have the same meaning as commonly understood to one of ordinary skill in the art.

The terms “target”, “target molecule”, and “target nucleic acid” are used interchangeable throughout the present specification. The term “target in a sample” refers to a molecule or nucleic acid in a sample, i.e. a molecule or nucleic acid to be analysed.

The term “nucleic acid” as used herein means a polymer composed of nucleotides, e.g. deoxyribonucleotides or ribonucleotides. The terms “ribonucleic acid” and “RNA” as used herein means a polymer composed of ribonucleotides. The terms “deoxyribonucleic acid” and “DNA” as used herein means a polymer composed of deoxyribonucleotides. The term “target nucleic acid” as used herein denotes single stranded nucleotide multimers of from about 10 to about 100 nucleotides up to about 1000 and more nucleotides in length.

The term “sample” within the context of the present invention may be virtually any sample, however, most usual refers to biological or biochemical samples. The term “biological sample,” as used herein, refers to a sample obtained from an organism such as humans, animals, plants, fungi, yeast, bacteria, viruses, tissue cultures or viral cultures or a combination of the above, or obtained from components (e.g., cells) of such an organism. The sample may be of any biological tissue or fluid. Frequently the sample will be a “clinical sample” which is a sample derived from a patient. Such samples include, but are not limited to, sputum, cerebrospinal fluid, blood, blood fractions such as serum including fetal serum (e.g., SFC) and plasma, blood cells (e.g., white cells), tissue or fine needle biopsy samples, urine, peritoneal fluid, and pleural fluid, or cells there from.

The term “oligonucleotide” refers to a molecule usually composed of 25 or fewer nucleotides. The term “detector oligonucleotide” as used within the present specification refers to oligonucleotides immobilized onto a solid support and usually composed of 20 nucleotides. The term “probe” within the present invention refers to a defined single-stranded nucleic acid (DNA or RNA) that is used to identify, usually through the use of a label, specific DNA or RNA molecules bearing the complementary sequence. Within the present invention, “ligation-mediated probes” are formed after ligation of adjacent first and second probes.

Within the methods of the present invention, the ligation-mediated probes are composed of a first and a second probe (see FIG. 1). The first probe comprises at its 5′ end a sequence of 10 to 50 nucleotides for hybridisation with a PCR primer and at its 3′ end a sequence of 10 to 50 nucleotides for hybridisation with a sample nucleic acid. The second probe comprises at its 5′ end a sequence of 10 to 50 nucleotides for hybridisation with a sample nucleic acid and at its 3′ end a sequence of 10 to 50 nucleotides for hybridisation with a PCR primer.

Either the first or the second probe additionally comprises a third sequence region or intermediate sequence which is located between the 5′ and the 3′ ends of the said first or second probe. The intermediate sequence (also called insert sequence) is 10 to 50 nucleotides long and consist of an artificial sequence composed of only three types of naturally occurring nucleotides and having no relation to the target or sample nucleic acids.

Within each first or second probe, the intermediate sequence part has the same fixed length but unique sequence of nucleotides, allowing a sequence-based identification and quantification of amplified ligation-mediated probes. As an alternative, the length of the intermediate sequences within different first or second probes may not be fixed and may differ between different probes. The use of only three types of nucleotides in designing the intermediate sequences provides the advantage that detection of the ligation-mediated probes can be done straightforward by microarray analysis (avoiding a less accurate detection based on the particular length of a ligation-mediated probe). The correspondence of the intermediate sequence parts with the detector oligonucleotide sequences provides the advantage that the respective 3′ end of the first probe and the 5′ end of the second probe may be changed to extend the scope of array analysis without the need of developing new detector oligonucleotide arrays.

Accordingly, in one embodiment of the present invention, a method is provided as described herein, wherein said intermediate sequence has a fixed length.

In a further embodiment of the present invention, a method is provided as described herein, wherein the fixed length of the detector oligonucleotides is between 10 and 50 nucleotides.

Either the first or second probe as used within the present invention is composed of an intermediate sequence part allowing the capture by a detector oligonucleotide on the solid support of ligation-mediated probes via said intermediate sequence part.

Said intermediate sequence part of the first or second oligonucleotide allows the capture of ligation-mediated probes without knowledge of the target sequence. As such the method according to the present invention may be easily extended with new first and second probes or markers without the need of developing new detector oligonucleotide arrays. The term “marker” as used within the present specification relates to a probe sequence corresponding to an identifiable physical location on a chromosome (for example, restriction enzyme cutting site, gene) whose inheritance can be monitored. Markers can be expressed regions of DNA (genes) or some segment of DNA with no known coding function but whose pattern of inheritance can be determined.

The present invention thus allows the multiplicity of first and second probes used in an experiment to be replaced by another multiplicity of probes without the need of developing a different array of detector oligonucleotides.

Accordingly, in one embodiment of the present invention, a method is provided as described herein, wherein the detector oligonucleotide are complementary to the said intermediate sequence part of the first or second probes.

The detector oligonucleotide design for use within the present invention is based on the selection of nucleotides out of only three types of naturally occurring nucleotides, e. g. only A, T and C; or A, C and G; or A, T, and G; etc. The term “naturally occurring nucleotides” includes deoxyribonucleotides and ribonucleotides. The particular restriction with respect to detector oligonucleotide design provides the advantage of minimizing cross-homology and increasing specificity. Cross-homology is defined as a length of a stretch of nucleotides in the detector oligonucleotide which overlaps with any of the other detector oligonucleotides and may be expressed as a percentage of the length of the detector oligonucleotide; or is defined as a length of a stretch of nucleotides in the detector oligonucleotide which overlaps with genomic sequences other than the original sequence wherefrom the detector oligonucleotide is derived from.

For use within the present invention, detector oligonucleotides are randomly selected and analysed for cross-homology against all other detector oligonucleotides for use on the same solid support.

In particular, the detector oligonucleotides within the present invention are designed according to a series of steps based on a random selection of non-eukaryotic gene sequences. The development of a totality of steps has provided the inventors of the present invention with a tool to design sets of unique detector oligonucleotide sequences.

The advantages of the random selection of artificial sequences include

(1) high specificity with no significant cross-homology amongst the detector oligonucleotides (homology<or=8 bases), no significant cross-homology between the detector oligonucleotides and PCR primer sequences (homology<or=4 bases) and no significant similarity (e-value: 0.01) in the detector oligonucleotides are found in the sequences of human, chimp, mouse and rat;

(2) high multiplicity wherein all of the detector oligonucleotides can simultaneously be used for detecting genomic information due to the unique sequence for each of the said detector oligonucleotides; and

(3) identical hybridization conditions wherein all of the detector oligonucleotides fulfil the same design criteria and thus identical hybridization conditions can be applied for all of DNA and RNA analysis.

Departing from a genomic sequences databank, e.g., the National Center for Biotechnology Information (NCBI) databank, the first step is a selection of a group of non-eukaryotic genomic sequences, e.g., nucleotide sequences from plant genomes. Secondly, a random selection is carried out of one non-eukaryotic genomic sequence in the selected group, e.g., a random selection of one plant genomic sequence. Then, an oligonucleotide, e.g. a sequence of 20 contiguous nucleotides, is again randomly selected out of the randomly chosen genomic sequence.

For these oligonucleotide sequences to be suitable within the present invention, the original 4-base composed sequence is converted to a new 3-base composed sequence by e.g. replacing each cytosine base in the selected sequence by a guanine base. The expressions “4-base” and “3-base” compositions as used within the present specification refer to sequences composed of respectively the four standard nucleotide bases adenine (A), thymine (T), guanine (G), and cytosine (C) and composed of only three out of these four nucleotide bases.

The obtained 3-base composition then undergoes internal rearrangements till certain postulated criteria are met. As such, unique artificial detector oligonucleotide sequences are designed being specific for hybridisation with the intermediate sequence within the first or second probe and having no cross-homology to target sequences in a sample. The expression “no cross-homology” as used in this context within the present specification means that cross homology is minimal and may have a value of up to 40% including 2%, 5%, 8%, 10%, 12%, 15%, 18%, 20%, 22%, 25%, 28%, 30%, 32%, 35%, 38%.

Most suitable detector oligonucleotides have a length of 20 nucleotides with a GC content of 55%, a melting temperature (Tm) of 68° C., an internal homology equal or less to 4 and having no secondary structure.

Other suitable detector oligonucleotides are characterized by values deviating within certain limits from the above values. For example, the GC content of the designed detector oligonucleotides may vary within a certain narrow range such that a strict common melting temperature remains guaranteed and identical hybridization conditions for all the detector oligonucleotides in the array is ensured. Accordingly the detector oligonucleotides within the present invention contain a GC content within the range of 5 to 75%. More suitable GC content ranges between 10 to 75%, 15 to 75%, 25 to 70%, 35 to 65%, 40 to 65%, and 45 to 60%. As mentioned above, a particular suitable GC content comes to 55%.

The steps of random selection of a group of non-eukaryotic genomic nucleotide sequences, subsequent random selection of a single non-eukaryotic genomic nucleotide sequence from the said group, random selection of an oligonucleotide nucleotide sequence and conversion of this sequence to a rearranged 3-base composed sequence is repeated to obtain a complete set of detector oligonucleotides that will eventually be arrayed on a solid support.

The detector oligonucleotide arrays of the present invention may be of any desired size; the detector oligonucleotides are usually arranged within spots or predefined regions. The upper and lower limits on the size of the support with respect to the number of spots are determined solely by the practical considerations of working with extremely small or large supports and may be from 2 spots to 10⁶ spots. Suitable arrays or microarrays within the present invention comprise between 50 and 400 spots. Within the present invention, usually an array of 124 different detector oligonucleotides or spots of detector oligonucleotides is employed.

Prior to arraying, each detector oligonucleotide is blasted against all other detector oligonucleotides belonging to the same group or set of oligonucleotides retained for immobilization within a same array on a same support. As such, each detector oligonucleotide is analysed for possible cross-homology against each and other oligonucleotide in a set, including the amplification forward and reverse primers. To this end, all sequences within a set are joined to form one contiguous sequence which is then analysed for cross-homology in respect of one oligonucleotide (e.g., 20-nucleotide) sequence and repeated for all other detector oligonucleotides within the joined sequence. During this intra-sequential homology analysis, a typical value in the range of 6-10 or less base pair cross-homology is maintained as a criterion for retaining a particular detector oligonucleotide. Usually, this criterion is lowered to a typical value in a range of 3-7 for the reverse and forward primers. A particular suitable value for allowed cross-homology of detector oligonucleotides is 8 and for reverse and forward amplification primers is 4.

Further, each detector oligonucleotide is analysed for cross-homology against other genomes such as human, chimp, mouse and rat genes present in the known databases. Each of the detector oligonucleotides was blast against all of the human, chimp, mouse and rat specific sequences using the databases of genome (all assemblies) and RefSeq RNA at http://www.ncbi.nlm.nih.gov. No significant similarity (e-value: 0.01) is found in the sequences of human, chimp, mouse and rat.

The detector oligonucleotides for use within the present invention contain a predicted cross-homology against other genomes of between 30 to 70%. The detector oligonucleotides for use within the present invention may contain a more suitable predicted cross-homology of between 40 to 60%. A particular suitable predicted cross-homology is less or equal to 50%.

Accordingly, in one embodiment of the present invention, a method is provided as described herein, wherein said detector oligonucleotides are random artificial sequences having the same GC content.

The detector oligonucleotides can be immobilized on the support using a wide variety of techniques. For example, the detector oligonucleotides can be adsorbed or otherwise non-covalently associated with the support (for example, immobilization to nylon or nitrocellulose filters using standard techniques); they may be covalently attached to the support; or their association may be mediated by specific binding pairs, such as biotin and streptavidin.

So-called spacer molecules may be useful in the application for spacing the detector oligonucleotides away from the solid support. Spacers may be long or short, flexible, semi-rigid or rigid, charged or uncharged, hydrophobic or hydrophilic, depending on the particular application. An example of a spacer suitable for use within the present invention is a 5T-spacer or 5-thymidine-spacer.

Accordingly, in one embodiment of the present invention, a method is provided as described herein, wherein said detector oligonucleotides each are immobilized onto the solid support via a spacer.

Other useful spacers which may be present on the surface of a solid support and used for attachment of detector oligonucleotides to said surface may also be bifunctional, i.e. having one functional group or moiety capable of forming a linkage with the solid support and any other functional group or moiety capable of forming a linkage with another spacer molecule or the detector oligonucleotide.

Regarding the samples, they may be analyzed directly or they may be subject to some preparation prior to use in the assays of this invention. Non-limiting examples of said preparation include suspension/dilution of the sample in water or an appropriate buffer or removal of cellular debris, e.g. by centrifugation, or selection of particular fractions of the sample before analysis. The method according to the present invention typically does not require pre-amplification of a nucleic acid sample. Within the methods of the present invention, first and second probes are allowed to directly hybridise on the sample nucleic acids. After ligation, the ligation-mediate probes are quantitatively amplified. The amount of the ligation-mediated probes represents the copy numbers of the target nucleic acids.

Accordingly, in one embodiment of the present invention, a method is provided as described herein, wherein the sample is non-amplified nucleic acid.

The target nucleic acids in a sample may comprise genomic DNA, genomic RNA, expressed RNA, microRNA (miRNA), plasmid DNA, mitochondrial or other cell organelle DNA, free cellular DNA, viral DNA or viral RNA, chemically pre-treated DNA, or a mixture of two or more of the above.

Accordingly, in one embodiment of the present invention, a method is provided as described herein, wherein said non-amplified nucleic acid is genomic DNA, genomic RNA, expressed RNA, microRNA (miRNA), plasmid DNA, mitochondrial or other cell organelle DNA, free cellular DNA, viral DNA or viral RNA, chemically pre-treated DNA, or a mixture of two or more of the above.

Particular interesting sample nucleic acids are composed of mutated DNA including naturally occurring mutations and induced mutations by mutagens. DNA has so-called hotspots, where mutations occur up to 100 times more frequently than the normal mutation rate. An example of a hotspot can be at an unusual base, e.g. 5-methylcytosine. 5-methylcytosine is the most frequent covalently modified base in the DNA of eukaryotic cells. The identification of 5-methylcytosine as a component of genetic information is of considerable interest in view of the fact that erroneous DNA methylation is an important factor in human disease because it contributes to tumorigenesis and ageing. 5-methylcytosine, however, cannot be identified by sequencing, since 5-methylcytosine has the same base-pairing behaviour as cytosine. In addition, in the case of PCR amplification, the epigenetic information, which is borne by 5-methylcytosines, is completely lost.

The usual methods for methylation analysis include e.g. the use of methylation-specific restriction enzymes. However, measuring patterns of cytosine methylation in genomic DNA is most efficiently accomplished by use of the bisulphite method wherein unmethylated cytosine residues are converted to uracil by hydrolytic deamination, but methylated cytosine residues remain unconverted (Grigg G. W., DNA Seq. 1996; 6(4):189-98; Paulin R et al, Nucleic Acids Res. 1998, 26(21):5009-10).

Accordingly, in one embodiment of the present invention, a method is provided as described herein, wherein the sample nucleic acids are chemically pretreated DNA wherein unmethylated cytosines are converted to uracil.

According to a further embodiment of the present invention, a method is provided as described herein, wherein said pretreated DNA is bisulfite-treated DNA.

The methods of the present invention can discriminate between two sequences that differ by as little as one nucleotide. Thus, the method of the invention can be used to detect a specific target nucleic acid molecule that has a mutation of at least one nucleotide. Preferably, said mutation is a single nucleotide polymorphism.

A variety of labels may be employed for the detection of the ligation-mediated probes within the present invention. The term label as used in the present specification refers to a molecule propagating a signal to aid in detection and quantification. Said signal may be detected either visually (e.g., because it has a coloured product, or emits fluorescence) or by use of a detector that detects properties of the reporter molecule (e.g., radioactivity, magnetic field, etc.). In the present specification, labels allow for the detection or the identification and quantification of nucleic acids within a sample. Detectable labels suitable for use in the present invention include but are not limited to any composition detectable by photonic, electronic, acoustic, opto-acoustic, gravity, electrochemical, electro-optic, spectroscopic, mass-spectrometric, enzymatic, immunochemical, chemical, photo-chemical, biochemical, optical or physical means

Accordingly, in one embodiment of the present invention, a method is provided as described herein, wherein said detectable labeled PCR primers allow detection by photonic, electronic, acoustic, opto-acoustic, gravity, electro-chemical, electro-optic, spectroscopic, mass-spectrometric, enzymatic, immunochemical, chemical, photo-chemical, biochemical, optical or physical means

Accordingly, virtually any label that produces a detectable, quantifiable signal and that is capable of being attached to a nucleotide and/or incorporated into the generated amplicon or amplified ligation-mediated probe can be used in conjunction with the methods of the present invention. Suitable labels include, by way of example and not limitation, radioisotopes, fluorophores, chromophores, chemiluminescent moieties, chemical labelling such as ULS labelling (Universal Linkage system; Kreatech) and ASAP (Accurate, Sensitive and Precise; Perkin Elmer), etc. Suitable labels may induce a colour reaction and/or may be capable of bio-, chemi- or photoluminescence.

Fluorescent labels are particularly suitable because they provide very strong signals with low background. Fluorescent labels are also optically detectable at high resolution and quick scanning procedure. Fluorescent labels offer the additional advantage that irradiation of a fluorescent label with light can produce a plurality of emissions. Thus, a single label can provide for a plurality of measurable events.

Accordingly, in one embodiment of the present invention, a method is provided as described herein, wherein said detectable label is fluorescent.

Desirably, fluorescent labels should absorb light above about 300 nm, usually above about 350 nm, and more usually above about 400 nm, usually emitting at wavelengths greater than about 10 nm higher than the wavelength of the light absorbed. Particular useful fluorescent labels include, by way of example and not limitation, fluorescein isothiocyanate (FITC), rhodamine, malachite green, Oregon green, Texas Red, Congo red, SybrGreen, phycoerythrin, allophycocyanin, 6-carboxyfluorescein (6-FAM), 2′,7′-dimethoxy-4′,5′-dichloro-6-carboxyfluorescein (JOE), 6-carboxy X-rhodamine (ROX), 6-carboxy-2′,4′,7′,4,7-hexachlorofluorescein (HEX), 5-carboxyfluorescein (5-FAM), N,N,N′,N′-tetramethyl-6-carboxyrhodamine (TAMRA), cyanine dyes (e.g. Cy5 and Cy3, including e.g. Oyster® dyes by Flownamics®, Madison), BODIPY dyes (e.g. BODIPY 630/650, Alexa542, etc.), green fluorescent protein (GFP), blue fluorescent protein (BFP), yellow fluorescent protein (YFP), red fluorescent protein (RFP), and the like, (see, e.g. Alexa dyes by Molecular Probes, Eugene, Oreg., USA; Dyomics, Germany).

Before microarray data are analysed, normalization methods are applied to remove unwanted biases present in the data that are obscuring the true detection and quantification of the sample nucleic acids. Within the method of the present invention, a fixed amount of control nucleic acid of known sequence is added to the sample.

Data from one or more arrays of detector oligonucleotides (microarrays) may be used per sample. Individual quantified signals of the amplified ligation-mediated probes hybridised on the detector oligonucleotides are normalized on the median signal of all amplified ligation-mediated probes hybridised on the array of detector oligonucleotides. Multiple microarray results can be used to minimize experimental noise and bias. For each of the target nucleic acid SNP one or more amplified ligation-mediated probes are used to detect and quantify different allelic variations of the target nucleic acid. The signal ratio of each amplified ligation-mediated probe for e.g. a particular SNP (e.g. A, C, G or T) or deletions or insertions is represented as a percentage of the sum of signals of all amplified ligation-mediated probes selected for a particular SNP. This percentage can be compared to a set of samples with known sequence composition.

As an alternative, amplified ligation-mediated probes derived from nucleic acid from a known source and composition can be labelled with a different label and mixed with differently labelled amplified ligation-mediated probes derived from nucleic acid from a sample of unknown composition. Both sets of amplified ligation-mediated probes are hybridised on the same microarray and results are obtained from two different labels. The ratio of the signals of known and unknown amplified nucleic acids may be used to normalize, detect and quantify the SNP or gene copy numbers.

Accordingly, in one embodiment of the present invention, a method is provided as described herein, wherein said quantification is by applying a normalization algorithm.

A number of materials suitable for use as solid support in the present invention have been described in the art.

The substrate may be in the form of beads, particles, sheets, films or membranes and may be permeable. For example, the substrate may consist of bead or particles (such as conventional solid phase synthesis supports), fibers (such as glass wool or other glass or plastic fibers), glass or plastic capillary tubes, porous supports or porous membranes. The solid support may be planar or have simple or complex shape. The surface to which the array of detector oligonucleotide probes is adhered may be the external surface or, in case of a porous solid substrate, the internal surface. Particularly where the solid support is porous, the detector oligonucleotide probes are likely to be attached to the internal surface.

Particular suitable materials for use as support in the present invention include any type of porous supports known in the art.

Within the present invention, the term “porous support” relates to any support capable of becoming penetrated by e.g. fluids, molecules, particles, gas, etc. The structure of a porous support may vary greatly and as such may be composed of e.g. a network of fibres or having pores, channels or capillaries going through the solid material. Within the present specification, the terms “pore”, “channel” and “capillary” are used interchangeable and refer to openings within the solid support which provide a porous character which may be flow-through or not.

Accordingly, in one embodiment of the present invention, a method is provided as described herein, wherein said solid support is a porous solid support.

The porous nature of the support member facilitates the pressurized movement of fluid, e.g. the sample solution, through its structure resulting in significantly reduced hybridisation times and increased signal and signal-to-noise ratios.

A porous support that may be penetrated through its entire thickness (i.e. having openings that open out on its top and bottom surface), is also referred to as a through-going or flow-through support. A positive or negative pressure may be applied to such supports in order to pump e.g. the sample solution dynamically up and down through the pores, channels or capillaries of the support.

By repeatedly applying a pressure difference over a flow-through support and thus forcing e.g. said sample solution to pass through the channels of the support and back results in a better mixing of ingredients, and is thus an alternative for mechanical mixing methods such as bubbling, stirring, vortexing or agitating. Such incubation dynamics also enables highly efficient reaction kinetics and washing efficiencies, resulting in high quality reaction products, because diffusion distances, which are rate limiting in other solid phase support materials (e.g. resins, gels, CPG and the like), are extremely short, and reaction components which have not been used are immediately and efficiently removed from the pore inner surface area can thus not interfere with subsequent handlings. Thus efficient use is made of the capillary forces of the pores, channels or capillaries in a flow-through support. The person skilled in the art will appreciate that the dimensions of the channels and the composition of the matter to be flown through and back provide a particular assessment of the pressures applied.

The porosity of such support may result from a multiplicity of essentially parallel pores, the pores being perpendicular to the upper and lower surfaces of the support. It will further be understood that the term “essentially parallel pores”, including through-going oriented channels, is not restricted to discrete channels, but also includes branched pores which are connected to adjacent pores in the substrate. As such, a particular useful porous solid support may have long branched or partially branched capillaries, which are interconnected inside the substrate, yet do not allow cross-communication between e.g. multiple samples spotted onto distinct areas on the support surface. The so-called interconnections allow for more precisely determining kinetic binding parameters which allow for an improved view on interaction behaviour in natural environments. Where matter may be flown forth and back through a porous support without lateral diffusion or cross-contamination and loss of matter within the porous structure, such porous support is likely to be a flow-through porous support having oriented through-going channels.

Accordingly, in one embodiment of the invention, a method is provided as described herein, wherein said porous solid support is a flow-through porous solid support.

The material of a porous support may be, for example, a metal, a ceramic metal oxide or an organic polymer. In view of strength and rigidity, a metal or a ceramic metal oxide may be used. Above all, in view of heat resistance and chemicals resistance, a metal oxide may be used. In addition, metal oxides provide a substrate having both a high channel density and a high porosity, allowing high density arrays comprising different first binding substances per unit of the surface for sample application. In addition, metal oxides are highly transparent for visible light. Metal oxides are relatively cheap supports that do not require the use of any typical microfabrication technology and that offers an improved control over the liquid distribution over the surface of the support, such as electrochemically manufactured metal oxide membrane. Metal oxide membranes having through-going, oriented channels can be manufactured through electrochemical etching of a metal sheet.

Accordingly, in one embodiment of the present invention, a method is provided as described herein, wherein said solid porous support is a metal-oxide support.

The kind of metal oxide is not especially limited, but can be preferably used. As a metal, for example, a porous substrate of stainless steel (sintered metal) can be used. For applications not requiring heat resistance, a porous substrate of an organic polymer can also be used if it is rigid.

Metal oxides considered are, among others, oxides of zirconium, silicium, mullite, cordierite, titanium, zeolite or zeolite analog, tantalum, and aluminum, as well as alloys of two or more metal oxides and doped metal oxides and alloys containing metal oxides.

Accordingly, in one embodiment of the present invention, a method is provided as described herein, wherein said metal oxide porous support is an aluminum-oxide porous support.

It is a further object of the present invention to provide for the use of a method as described herein, for detecting nucleotide variations in a nucleic acid sample, said variations selected from the group comprising deletions and insertions, including frame-shift mutations; and base-pair substitutions, including single nucleotide mutations or polymorphisms.

An example of the use of the methods according to the present invention is the analysis of mitochondrial DNA (mtDNA) mutations (see Example 1 below).

As a further example, the methods according to the present invention are also suitable for RNA detection methods. For example, the methods according to the present invention are particularly useful for detection of microRNA.

During the past years, hundreds of genes that encode small RNA molecules have been discovered. These so-called microRNAs (miRNA) are small single-stranded RNAs of about 22 nucleotides long and play important roles in plants and animals. For example, there are hints that the levels of some miRNAs are altered cancer. Much attention is currently drawn to the identification and quantification of these miRNAs in view of their potential as clinical diagnostic tools.

The methods according to the present invention provide an efficient and multiplexed detection of these miRNAs. In particular the multiplexed detection within the methods of the present invention provides an important advantage over methods in the art, e.g., the method described by Lao et al (Biochemical and Biophysical research Communications 343, 85-89, 2006) wherein the detection is singleplex.

Multiplex ligation-dependent probe amplifications performed using the methods of the present invention for detection of microRNA departs with a preparative step wherein the target molecules or the miRNA is first linked at their 3′ end to an artificial sequence or spacer unique for each miRNA. This is because due to their length, miRNA typically bear length constraints in respect of primer-aided amplification.

These artificial spacers are included within the joined contiguous sequence as mentioned above including reverse and forward amplification primers and all detector oligonucleotides of a particular set for cross homology analysis. A typical value ranging from 6-10 or less base pair cross-homology is a criterion for retaining a particular spacer. A more convenient cross-homology criterion has a value of 8 base-pairs.

Following a complementary DNA synthesis step using reverse transcriptase (cDNA synthesis step), the miRNA-spacer conjugated molecules then may serve as target nucleic acid sequences in a sample within the methods of the present invention.

Further, the methods according to the present invention may also find use in multiplex detection of mRNA expression. Hereto, target mRNA molecules are first ligated at their 3′ end to a reverse amplification primer sequence comprising 3′ a universal sequence for later amplification as indicated in step (v) of the method as disclosed in claim 1. Following cDNA synthesis, the cDNA molecules are contacted with a forward primer composed of a 5′ end sequence part for hybridisation to a PCR primer, a 3′ end sequence part complementary to the 5′ and of the cDNA and an intermediate sequence for detection of the target sequence (cDNA) by hybridisation to an array of detector oligonucleotides according to the present invention. Following amplification with the forward primer, further PCR amplification of the obtained sequences and subsequent detection according to the methods of the present invention can be performed.

It is a further object of the present invention to provide microarrays for performing a method as described herein (eg according to any of claims 1 to 32), comprising a solid support, said solid substrate having immobilized thereon an array of detector oligonucleotides, wherein each detector oligonucleotide is composed of only three types of nucleotides and wherein said detector oligonucleotides are random artificial sequences having the same GC content.

In one embodiment, such a microarray is provided, wherein said solid support is a porous solid support.

In one embodiment, such a microarray is provided, wherein said porous solid support is a flow-through porous solid support.

In one embodiment, such a microarray is provided, wherein said solid porous support is a metal-oxide support.

In one embodiment, such a microarray is provided, wherein said metal oxide porous support is an aluminum-oxide porous support.

It is a further object of the present invention to provide a kit for performing a method as described herein, comprising:
(a) a microarray comprising an array of detector oligonucleotides as described herein;
(b) at least one set of first and second probes as described herein,
(c) a ligase for use in the formation of ligation-mediated probes;
(d) a set of PCR primers wherein the forward primer is complementary to the 5′ end of the first probe and the reverse primer is complementary to the 3′ end of the second probe as described herein.

An example of a suitable ligase is T4 RNA Ligase which is an ATP-dependent ligase, active on a broad range of substrates including RNA, DNA, oligoribonucleotides, oligodeoxynucleotides, as well as numerous nucleotide derivatives. The enzyme catalyzes the formation of a phosphodiester bond between a 5′-phosphoryl-terminated nucleic acid donor to a 3′-hydroxyl-terminated nucleic acid acceptor in a template-independent manner.

### DESCRIPTION OF FIGURES

FIG. 1 illustrates the formation of a ligation-mediated probe according to the present invention: a first probe (1) is composed of a 3′ end sequence (3′) for hybridisation to a target nucleic acid (T), and a 5′ end (5′) for hybridisation with a PCR primer. An intermediate sequence (IS) is present in between the 5′ end and the 3′ end of the first oligonucleotide. A second probe (2) is composed of a 5′ end (5″) for hybridisation to the target nucleic acid (T) and a 3′ end (3″) for hybridisation with a PCR primer. Said intermediate sequence may also be present within the second probe. The second probe (2) is characterised by having a 5′ phosphate group allowing ligation with a 3′ hydroxyl group at the first probe at the ligation site (LS).

FIG. 2 illustrates the process of hybridisation, ligation and amplification;

FIG. 2A shows the hybridization of the first and second probes to the target nucleic acids under conditions favouring hybridisation;

FIG. 2B shows the ligation of the hybridised first and second probes to form a ligation-mediated probe;

FIG. 2C shows the quantitative amplification of the ligation-mediated probes using a set of detectable labelled PCR primers. The amount of PCR product from each of the probes is proportional to the copy number of the target nucleic acids.

FIG. 3 illustrates the detection and quantification on a porous solid support

FIG. 3A shows the immobilisation of detector oligonucleotides on a solid support (SS). In this example, an aluminum-oxide support was used as a support material. The support contains millions of pores (0.2×60 micron) in parallel orientation connecting the top and bottom surfaces.

FIG. 3B is a schematic depiction of immobilized detector oligonucleotides. The arrow indicates 200 nm.

FIG. 3C illustrates a flow-through hybridisation. Left: the PCR sample is pumped back and forth by air pressure through the porous substrate during the incubation. Right: a raw image acquired on an aluminum-oxide support. A Tiff image is recorded through the entire porous structure by an epi-fluorescent CCD imaging system. The image information is quantified by applying a normalisation algorithm. The horizontal arrow indicates the width of a aluminium-oxide pore of 200 nm; D, detector oligonucleotide.

FIG. 4 illustrates a mutation analysis of 27mtDNA samples using the present invention as described in Example 1.

FIG. 4A illustrates raw images which were obtained on a solid support comprising an array of 124 detector oligonucleotides by hybridisations of the ligation-mediated probes derived from two mtDNA samples S1 and S2. In this experiment as set out in Example 1, 70 sets of first and second probes were used to detect 33 known mtDNA mutations. The images were acquired at 1000 ms using Cy5 filter.

FIG. 4AA illustrates raw 12-bit Tiff images obtained from four hybridizations on PamArray using 5 ul of the amplified ligation-mediated probes from mitochondrial DNA sample 1, 5, 8 and 15. The images were recorded at 1000 ms using a Cy5 filter set. 124 detector oligonucleotides were spotted in duplicate along with reference and negative controls.

FIG. 4B illustrates the analysis of 27 mtDNA samples with known mutations on a solid support comprising an array of 124 detector oligonucleotides by hybridisations of their ligation-mediated probes. The figure shows signal clustering for all of the 27 samples. Clearly, two clustering groups were identified. A large part of the 27 samples did not give signal at the mutant sequences (top, light grey colour). Another large part of the samples had signals at the normal sequences (bottom, dark grey colour). The arrow indicates low to high signal.

FIG. 4C illustrates the allele % of 4 samples S5 to S7. A mutation analysis was performed on four mtDNA samples S5 (S5_{—}3302_A-G), S6 (S6_{—}3460_G-A), S7 (S7_{—}4269_A-G) and S8 (S8_{—}8344_A-G). Of the four samples, two known mtDNA mutations (A3302G in sample 5 and G3460A in sample 6) were reliably detected and quantified as allele frequency. Other two samples had wide type alleles at the two loci. The sample names given above are explained as follows: e.g., S5_{—}3302_A-G: S5 indicates the sample 5; 3302_A-G indicates that the sample 5 has a mutation with A3302G (base substitution from A to G at position 3302 position). The legend to the graphic reads as follows: e.g., Cp18-3302A_{—}5: Cp18 indicates the complementary sequence to Pam oligonucleotide 18 (detector oligonucleotide 18); 3302A indicates the base (A) at position 3302 in the mitochondrial genomic DNA (Genbank: V00662DNA); 5 indicates the sample 5 which has a mutation at the position of base 3302.

FIG. 4D illustrates the allele % of 4 samples S9 to S12. A mutation analysis was performed on four mtDNA samples S9 (S9_{—}8993_T-CG), S10 (S10_{—}9176_T-C), S11 (S11_{—}10750_G-A), and S12 (S₁₂_{—}11778_G-A). Of the four samples, one known mtDNA mutation T8993G (8993_T-C-G-A) in sample 9 was detected and quantified. The other three samples had only wild type allele at this locus. The sample names above can be explained similarly as set out for FIG. 4C. The legend to the graphic is similar as explained for FIG. 4C.

FIG. 5 illustrates the layout of an array of 124 detector oligonucleotides. The detector oligonucleotides were spotted in duplicate along with reference and exogenous oligonucleotides. Light grey colour indicates reference oligonucleotide. Dark grey colour indicates exogenous oligonucleotide (Ambion 2) as a negative control. White colour indicates the detector oligonucleotides (Pam 1-124).

FIG. 6 illustrates the PamStation™ 4 system.

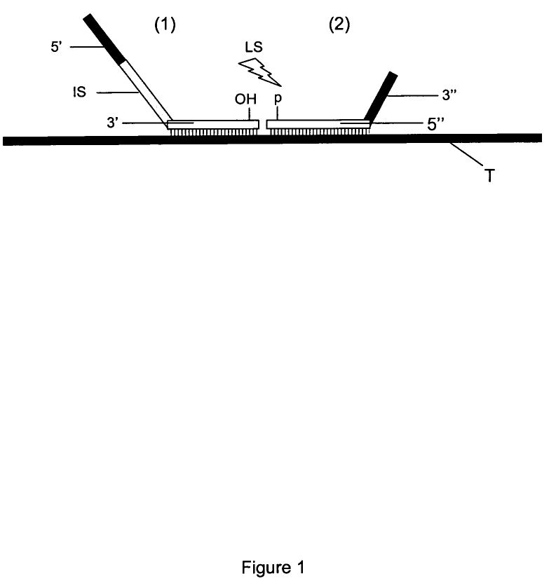

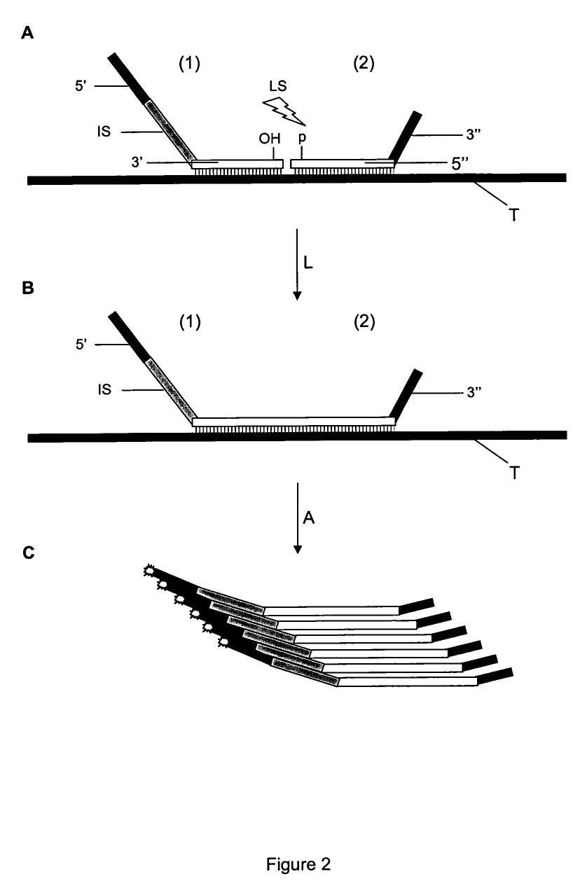

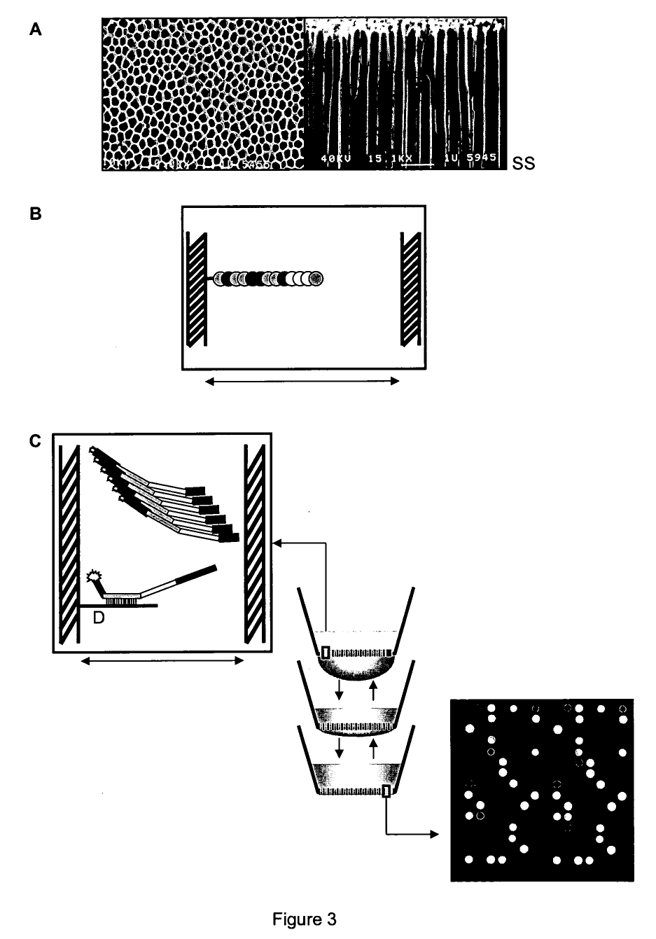

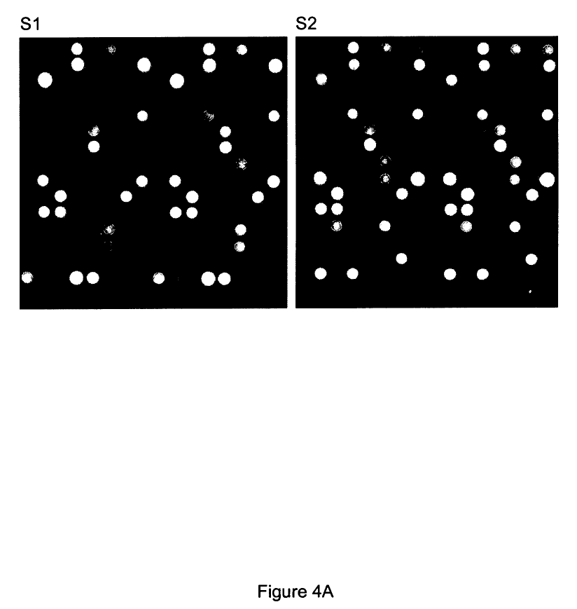

### EXAMPLES

The following examples of the invention are exemplary and should not be taken as in any way limiting.

### Example 1

### Assessment of the Feasibility for Analyzing Mitochondrial DNA (mtDNA) Mutations by Hybridisation of Ligation-mediated Probes on an Array with Detector Oligonucleotides

Material and Methods

1 Design of the Detector Oligonucleotides

The sequences of 124 detector oligonucleotides were designed based on the selection of artificial sequences with only three types of nucleotides. The design parameters were as follows:
1. Size: 20 bases;
2. % GC of probe 55;
3. Tm of probe: 68 (° C.);
4. Homology within the detector: between 2 and 4 bases;
5. Homology amongst the 124 detectors: between 4 and 8 bases; and
6. No significant similarity (homology less than 50%) blast against the human, chimp, mouse and rat specific sequences using the databases of genome (all assemblies) and RefSeq RNA at http://www.ncbi.nlm.nih.gov.

A 5T spacer was attached at the 5′ end of the oligonucleotides for each of 124 detector oligonucleotides.

2 Design of the Detector Oligonucleotide Array

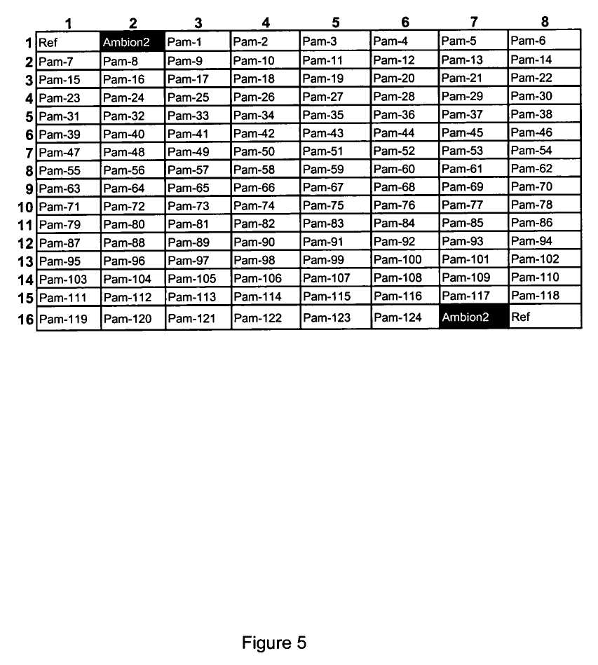

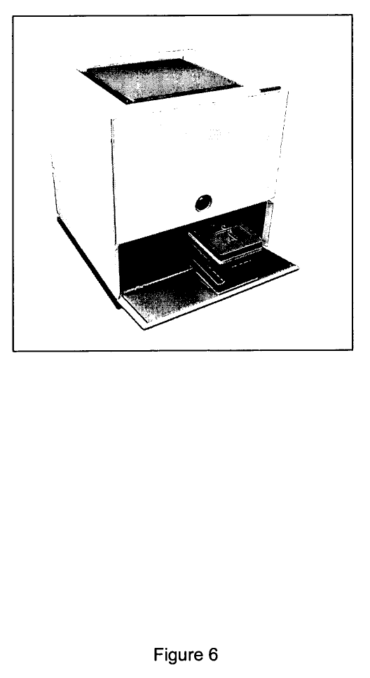

Preparation of the detector oligonucleotide array was performed as previously described (Van Beuningen et al., Clin. Chem., 47, 1931-1933, 2001; Wu et al., Nucleic Acids Res. 32, e123, 2004). The array (SO-0311PC4M) used in this study contained 128 features that were spotted in duplicate (256 spots). The 128 features consisted of 124 25-mer oligonucleotides, 2 oligonucleotides corresponding to ArrayControl RNA Spikes (Ambion), and 2 reference oligonucleotides. The spot layout of the array is shown in FIG. 5.

3 Design Ligation-mediated Probes

For analysing 33 of known mutations in mitochondrial DNA, 70 sets of ligation-mediated probes were designed including 4 control probes from D-loop region in genomic mitochondrial DNA. For each of the mutations, two sets of probes were designed (one for detection of normal allele and another one for mutant allele). The ligation mediated probes consist of one pair of probes characterized by: 1) A first probe is composed of a 5′ end sequence part (20 bases) for hybridisation to a PCR primer and a 3′ end sequence part (20-30 bases) for hybridisation to the target nucleic acid; 2) A second probe is composed of a 5′ end sequence part (20-30 bases) for hybridisation to the target nucleic acid, and a 3′ end sequence part (20 bases) for hybridisation with a PCR primer. 3) An intermediate sequence (20 bases) complementary to the detector oligonucleotide on the array is present in between 5′ and 3′ end sequence parts of the first probe. 4) A 5′ phosphate group is attached to 5′ end sequence of the second probe. The probes were synthesized by Illiumina (USA) without purification.

4 Hybridisation, Ligation and Amplification of Mitochondrial DNA Samples

100 ng of genomic mitochondrial DNA for each of 27 samples and 4 fM probe-mixture (70 sets of probes) was used as input for hybridisation, ligation and amplification with EK5 kit from MRC-Holland according to the protocol in the DNA analysis Manual. During amplification, the PCR products of the ligation-mediated probes were labelled using a forward primer with Cy5 modification.

5 Hybridisation and Detection

Hybridization, washing and detection were performed using a PamStation™ 4 system (FIG. 6). Prior to hybridisation, 5 μl of each PCR products was mixed with hybridisation buffer (1×SSPE and 0.1% N-Laurylsarcosine) with a final volume of 25 μl. Before hybridisation, the sample was denatured at 95° C. for 2 minutes and kept on ice until hybridisation. The sample was hybridised to the array of detector oligonucleotides as described above in 20 μl volume at 45° C. for 10 minutes under constant pumping (5 cycles/min). Subsequently, the arrays were washed at 55° C. with 1×SSPE/0.1% N-Laurylsarcosine for three times. After washing, two images were taken at 500 and 1000 ms using a Cy5 filter set.

6 Data Analysis

The image information was converted into spot intensity values using a customized 5.6 version of ImaGene (Biodiscovery). Median signal intensity and local background measurements were obtained for each spot on the hybridised array. Local background was subtracted from the value of each spot on the array. The signal intensity after background subtraction was used for further analysis. To assess the variability of hybridisations, the coefficients of variation (CV) for all of the probes from three different arrays were determined. A cut-off value for a positive signal was defined as above 10 AU of the mean signal.

For quantification of the target nucleic acids, individual quantified signals of the amplified ligation-mediated probes hybridised on the detector oligonucleotide are normalized on the median signal of all amplified ligation-mediated probes hybridised on the array. The results from triplicate hybridisations were used to minimize experimental noise and bias. For each of the target nucleic acid SNP, two or more amplified ligation-mediated probes were used to detect and quantify different allelic variations of the target nucleic acid. The signal ratio of each amplified ligation-mediated probe for a particular SNP (e.g. A, C, G or T) or deletions or insertions is represented as a percentage of the sum of signals of all amplified ligation-mediated probes selected for a particular SNP. This percentage was compared to a set of samples with known sequence composition.

Results

1 Specificity and Reproducibility of Hybridization

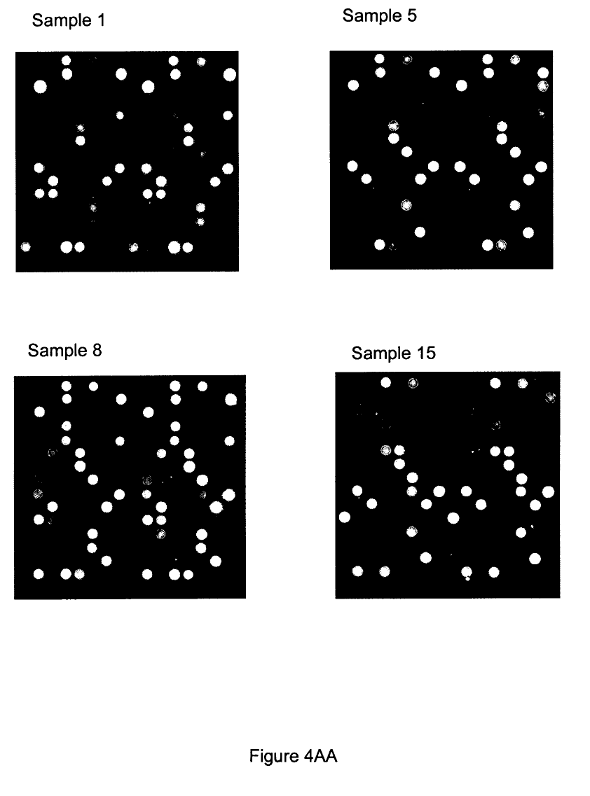

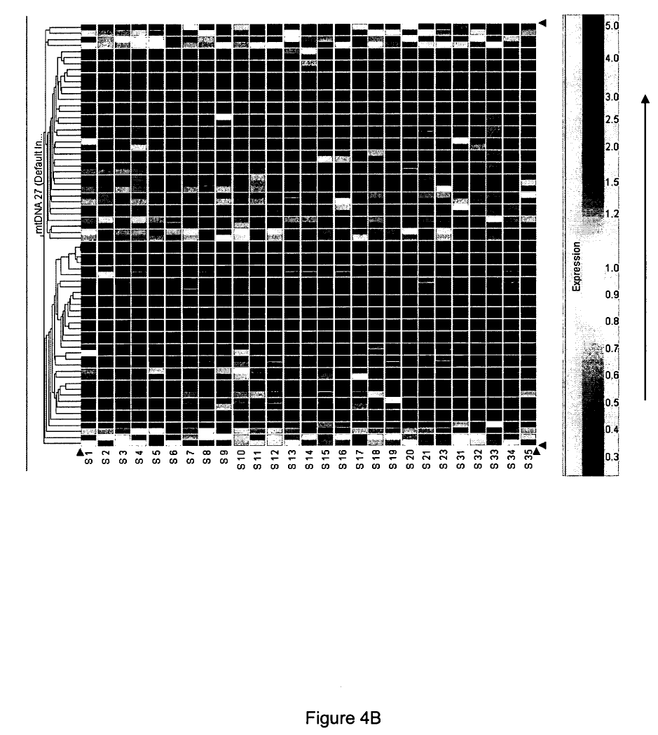

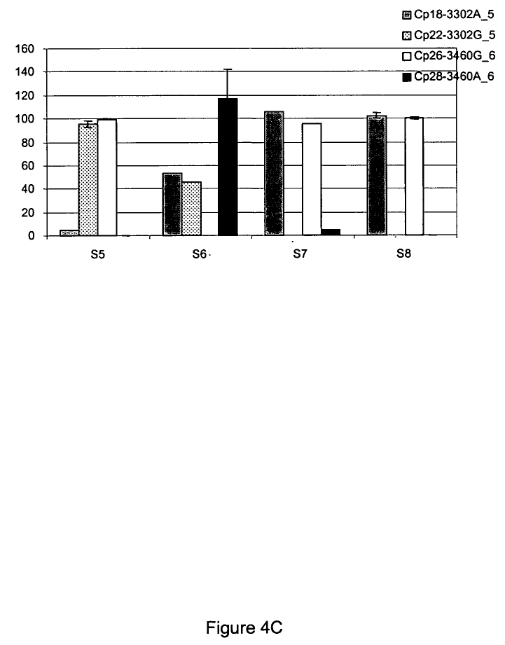

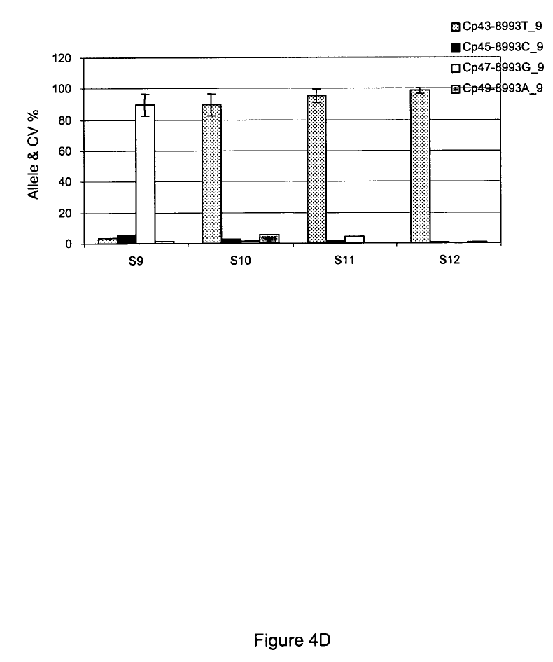

To determine the variability of hybridization, triplicate hybridizations for each sample were performed on three different 4-array disposables. The coefficient of variation (CV) for each individual spot was calculated based on the signals across the replicates. The average variability (CV) of signal level above 10 AU across 81 arrays was 8%. The average variability (CV) of allele frequency was 4%. The results indicate that the data acquired on the arrays are reproducible. Examples of raw images obtained from four different samples are shown in FIGS. 4A and 4AA. Of the 70 specific ligation-mediated probes for mitochondrial DNA, 35 (50%) gave rise to signals. The large majority of the samples did not give signal at the mutant sequences (FIG. 4B).

These results indicate that the hybridizations on the PamArray are specific.

2 Clustering Analysis of Signal Intensity using GeneSpring

To assess the feasibility for analyzing mitochondrial DNA mutations by hybridization of ligation-mediated probes on an array with detector oligonucleutides, signals obtained from all of hybridizations for 27 samples were imported into GeneSpring and clustered using standard correlation of gene tree. The signal patterns of the hybridizations among the 27 samples are displayed in FIG. 4B. Clearly, two clustering groups were identified among 70 of the ligation-mediated probes. A large part of the 27 samples did not give rise to a signal at the mutant sequences (upper part, light grey colour). Another large part of the samples gave rise to signals at the normal sequences (lower part, dark grey colour).

3 Mutational Analysis of Mitochondrial DNA

Using the present invention, all of the known point mutations in mitochondrial DNA were reliably detected and quantified as allele frequency. Two examples of the mutation analysis are shown in FIGS. 4C and 4D.

In FIG. 4C the allele % of 4 samples S5 to S8 is summarized. A mutation analysis was performed on four mtDNA samples S5 (S5_{—}3302_A-G), S6 (S6_{—}3460_G-A), S7 (S7_{—}4269_A-G) and S8 (S8_{—}8344_A-G). Of the four samples, two known mtDNA mutations (A3302G in sample 5 and G3460A in sample 6) were reliably detected and quantified as allele frequency. The other two samples had wide type alleles at the two loci.

In FIG. 4D the allele % of 4 samples S9 to S12 is summarized. A mutation analysis was performed on four mtDNA samples S9 (S9_{—}8993_T-CG), S10 (S10_{—}9176_T-C), S11 (S11_{—}0750_G-A), and S12 (S12_{—}11778_G-A). Of the four samples, one known mtDNA mutation T8993G (8993_T-C-G-A) in sample 9 was detected and quantified. The other three samples had only the wild type allele at this locus.

1-25. (canceled)

25. A method for multiplex detection and quantification of target nucleic acid sequences in a sample comprising the steps of:
(i) providing a solid support having immobilized thereon an array of detector oligonucleotides, wherein said array of detector oligonucleotides is designed by random selection of non-eukaryotic genomic sequences followed by random selection of 20-mer oligonucleotide sequences and subsequent conversion of these oligonucleotide sequences such that these are artificial sequences composed of only three types of nucleotides;
(ii) providing a sample having added thereto a fixed amount of control nucleic acid of known sequence;
(iii) contacting said sample with at least two probes that hybridize to adjacent sites of a target sequence under conditions favoring hybridization between the sample nucleic acids and the said at least two probes, wherein
a. a first probe is composed of a 5′ end sequence part for hybridization to a PCR primer and a 3′ end sequence part for hybridization to the target nucleic acid; and
b. a second probe is composed of a 5′ end sequence part for hybridization to the target nucleic acid, and a 3′ end sequence part for hybridization with a PCR primer; and
c. an intermediate sequence is present in between said 5′ and 3′ end sequence parts of said first or second probe; and
d. said second probe is characterized by having a 5′ phosphate group allowing ligation with a 3′ hydroxyl group at the said first probe forming a ligation-mediated probe;
(iv) ligation of the said hybridized first and second probes to form ligation-mediated probes;
(v) contacting a set of detectable labelled PCR primers with the ligation-mediated probes allowing amplification thereof;
(vi) detection and quantification of sample nucleic acids via hybridization of the said intermediate parts within the amplified ligation-mediated probes onto the array of detector oligonucleotides provided in (i).

26. A method according to claim 25, wherein said detector oligonucleotides are complementary to the said intermediate sequence part of the first or second probes.

27. A method according to claim 25, wherein said detector oligonucleotides have the same GC content.

28. A method according to claim 25, wherein said detector oligonucleotides each are immobilized onto the solid support via a spacer.

29. A method according to claim 25, wherein said sample is non-amplified nucleic acid.

30. A method according to claim 29, wherein said non-amplified nucleic acid is genomic DNA, genomic RNA, expressed RNA, microRNA (miRNA), plasmid DNA, mitochondrial or other cell organelle DNA, free cellular DNA, viral DNA or viral RNA, chemically pretreated DNA, or a mixture of two or more of the above.

31. A method according to claim 30, wherein said chemically pretreated DNA is DNA wherein unmethylated cytosines are converted to uracil.

32. A method according to claim 30, wherein said pretreated DNA is bisulfite-treated DNA.

33. A method according to claim 25, wherein said intermediate sequence has a fixed length.

34. A method according to claim 33, wherein said fixed length is between 10 and 50 nucleotides.

35. A method according to claim 25, wherein said detectable labeled PCR primers allow detection by photonic, electronic, acoustic, opto-acoustic, gravity, electro-chemical, electro-optic, spectroscopic, mass-spectrometric, enzymatic, immunochemical, chemical, photochemical, biochemical, optical or physical means.

36. A method according to claim 25, wherein said detectable label is fluorescent.

37. A method according to claim 25, wherein said quantification is by applying a normalization algorithm.

38. A method according to claim 25, wherein said solid support is a porous solid support.

39. A method according to claim 38, wherein said porous solid support is a flow-through porous solid support.

40. A method according to claim 29, wherein said solid porous support is a metal-oxide support.

41. A method according to claim 40, wherein said metal oxide porous support is an aluminum-oxide porous support.

42. A microarray for performing a method according to claim 25, comprising a solid support, said solid substrate having immobilized thereon an array of detector oligonucleotides, wherein each detector oligonucleotide is composed of only three types of nucleotides and wherein said detector oligonucleotides are random artificial sequences having the same GC content.

43. A microarray according to claim 42, wherein said solid support is a porous solid support.

44. A microarray according to claim 43, wherein said porous solid support is a flow-through porous solid support.

45. A microarray according to claim 43, wherein said solid porous support is a metal-oxide support.

46. A microarray according to 45, wherein said metal oxide porous support is an aluminum-oxide porous support.

47. A kit comprising:
(a) a microarray comprising an array of detector oligonucleotides according to claim 42;
(b) at least one set of first and second probes for hybridization to a sample nucleic acid,
(c) a ligase for use in the formation of ligation-mediated probes,
(d) a set of PCR primers which are complementary to the 5′ end of the first probe and the 3′ end of the second probe.
